(19) United States
(12) Patent Application Publication
(10) Pub. No.: US 2005/0153452 A1
(43) Pub. Date: Jul. 14, 2005

(54) Compositions for detecting food spoilage and related methods

(75) Inventors: John R. Williams, Lexington, MA (US); Kathleen E. Myers, Boston, MA (US)

Correspondence Address: GOODWIN PROCTER LLP;PATENT ADMINISTRATOR, 53 STATE PLACE, BOSTON, MA, 02109-2881, US

(73) Assignee: The Charles Stark Draper Laboratory, Inc., Cambridge, MA (US)

(21) Appl. No.: 11/035,236

(22) Filed: Jan. 13, 2005

Related U.S. Application Data
(60) Provisional application No. 60/536,110, filed on Jan. 13, 2004.

(51) Int. Cl. G01N 33/02

(52) U.S. Cl. 436/2; 436/20; 422/56

(57) ABSTRACT

Indicators for detecting food spoilage and related methods utilize a matrix having at least one surface for establishing fluid communication with a food to be monitored, and, physically associated with (e.g., entrained within or bonded to) the matrix, an amine-responsive compound that itself comprises or consists of a betalain (or derivative thereof), a flavonoid (or derivative thereof), or a combination of these.

### RELATED APPLICATION

This application claims the benefits of and priority to U.S. Ser. No. 60/536,110, filed on Jan. 13, 2004, the entire disclosure of which is hereby incorporated by reference.

### FIELD OF THE INVENTION

The present invention relates to monitoring of food quality, and in particular to compositions that undergo an observable color change in the presence of amines or other food degradation products.

### BACKGROUND OF THE INVENTION

Monitoring the quality of perishable food is a critical task throughout the food production and distribution chain. Many food products are subject to spoilage, as a result of improper handling, contamination or simply due to aging. If a perishable product such as meat is exposed to excessive temperatures during transit, for example, it will age and spoil prematurely, but ultimately spoilage is inevitable. Today, food distributors typically apply expiration dates to their products, but these dates essentially represent an estimate—that is, they assume an average (or even perfect) “heat history” that corresponds to a known aging profile. Except on a spot basis, food distributors generally do not continuously monitor the quality of their products.

Reasons for this include the complexity and expense of the laboratory-grade equipment typically needed to detect spoilage, the skilled manpower necessary to operate such equipment, and the need to obtain physical access to the food in order to run the test and cost. Monitoring food quality on an ongoing basis might require repeated penetration of the packaging in order to perform testing, each time followed by the need to repackage the food.

### BRIEF SUMMARY OF THE INVENTION

The present invention provides a simple and effective approach to determining the quality of food products without the need for repeated tests or to damage the original food packaging. The invention is responsive to volatile bases, particularly amines, generated by bacterial decomposition of proteins. In preferred embodiments, the invention utilizes one or more indicators comprising or derived from naturally occurring compounds such as betalains (which include betanidin, betacyanins, and betaxanthins) and/or flavonoids (which include anthocyanins and anthocyanidins) as detection chromophores; these compounds undergo a color change in the presence of amine compounds, and this color change is employed as an indicator of food quality. In general, the invention comprises a system for immobilizing an amine-responsive, naturally occurring compound (or derivative) and exposing it to food to be monitored, ideally in conjunction with ordinary food packaging.

For some foods and beverages, acid products are formed as the food spoils. For example, lactose in milk is converted to lactic acid and ethanol in wine is converted to acetic acid (vinegar). The same indicators used to detect bases such as amines may be employed to detect acid degradation products as well. This may be accomplished either by utilizing an alternate transition point if one exists, or by adjusting the pH of the indicator to observe the reverse of the change observed for amines. In this way, the indicator system provides an ongoing visual indication of food quality.

In some embodiments, the system is utilized as a vapor sensor, not directly contacting the food, in which case the naturally occurring compound may or may not be immobilized.

In some embodiments, the indicator is applied to or associated with the packaging, e.g., in the form of a label or as part of a cap (e.g., in the case of milk), or as part of the packaging itself (e.g., chemically integrated within a polymer wrap or container). The indicator is in direct contact or fluid communication with the food to be monitored or is used as a vapor sensor. Consumers may judge the quality of the food by comparing the color of the indicator to a reference chart supplied with the food (and ideally located adjacent to the indicator), which illustrates color shadings and the food quality level to which they correspond. Alternatively, the indicator color may be read photometrically, e.g., using a color densitometer, in order to provide a more precise reading of sensed amine levels. This latter approach may be employed by food suppliers not wishing to risk human error in discerning the quality of the food they sell. Color densitometers may take the form of simple hand-held units carried by, for example, store employees and stock clerks who routinely handle and shelve food products.

A variety of other readouts is possible; for example words or symbols may be printed using the color-changing indicator as ink. The ink may be printed on a clear or white background or on a colored background where the colored background is non-indicating (i.e., a fixed color). If the color of the background matches the initial color of the indicator, then letters or symbols will appear as the food quality deteriorates. The readout color can also be modified for visibility or aesthetic purposes.

Accordingly, in a first aspect, the invention comprises an indicator for detecting food spoilage. The indicator comprises a matrix having at least one surface for establishing fluid communication with a food to be monitored, and, immobilized within (e.g., by entrainment or chemical bonding) the matrix, an amine-responsive compound that itself comprises or consists of a betalain (or derivative thereof), a flavonoid (or derivative thereof), or a combination of these. In some preferred betalain embodiments, the indicator comprises or consists of an ester of betanin. In some preferred flavonoid embodiments, the indicator comprises or consists of anthocyanin or a derivative thereof, or anthocyanidin or a derivative thereof, or a combination of these.

The matrix may be a hydrophobic paper (e.g., silicone-treated filter paper), hydrophilic paper, hydrophilic paper with a hydrophobic coating, or a polymer matrix. The indicator compound(s) may be entrained within the polymer matrix or covalently bonded to the backbone of the polymer. In some embodiments, the matrix comprises clear gelatin. In other embodiments, the matrix comprises a colored gelatin to improve visibility of the indicator.

In a second aspect, the invention comprises a method of making an indicator for detecting food spoilage. The method comprises providing an indicator compound comprising a betalain or derivative thereof and/or a flavonoid or derivative thereof, and associating the compound with a matrix having at least one surface for establishing fluid communication with a food to be monitored. A color change indicates the degree, if any, of spoilage.

In a third aspect, the invention comprises a method of detecting food spoilage using a matrix having, associated therewith, an amine-responsive compound comprising a betalain or derivative thereof or a flavonoid or derivative thereof. The method comprises establishing fluid communication between the matrix and a food to be monitored. The amine-responsive compound changes color in response to amines or acids present in or generated by the food, and observing the color change facilitates detection of food spoilage.

### DETAILED DESCRIPTION OF THE PREFERRED EMBODIMENTS

Betalains suitable for use in connection with the present invention are red-violet betacyanins that accumulate naturally in flowers, fruits and some vegetables, most notably beets. Useful compounds include betanidin, betanin and their derivatives.
These have the chemical formula 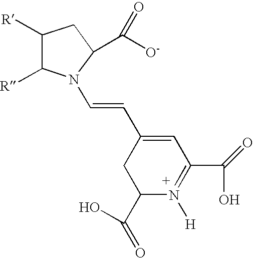
where R′=R″=OH for betanidin and, for betanin, R′=GlcO (where Glc refers to glucose) and R″=OH. The identities of R′ and R″ are not critical to the invention, however, and may be hydrogen atoms or other substituents.

In a preferred embodiment, the carboxyl groups are esterified. For example, ester derivatives of betanin can be prepared by reaction with an alcohol in the presence of a strong acid, such as sulfuric acid:

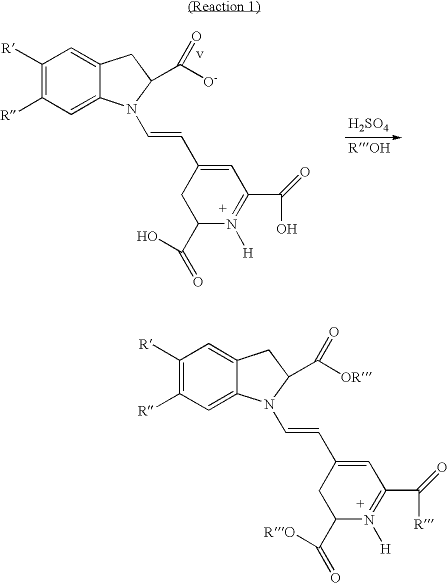

In preferred embodiments, R′ and R″ are OH, ester, alkyl, aryl, or mixed alkyl-aryl groups, or GlcO, and R′″ is an alkyl, aryl, or mixed alkyl-aryl group. In order to prevent gradual loss of indicator activity due to oxidation, it may be desirable to utilize R′ and R″ groups lacking moieties subject to oxidation. Antioxidants may also be employed in the formulation. In some embodiments, R′″ is an alkyl group having from one to 20 carbon atoms, and may be linear, branched, cyclic, or a combination. In other embodiments, R′″ may be an aryl compound based, for example, on aromatic rings having one, two or three members.

In one experiment, beet juice, a source of betanin, was reacted with methanol. In a 250 ml Erlenmeyer flask, 10 grams of beet juice extract and 200 ml of methanol were stirred at 25° C. To the red solution was added 1 ml of sulfuric acid. The solution was stirred for 4-6 hrs during which time the solution changed from red to purple; the change was accompanied by the appearance of an absorbance in the IR spectrum at 1735 cm⁻¹. When Whatman PS paper was dipped into the resultant solution and dried, the indicator remained on even when rinsed under running tap water for a minute.

Flavonoids suitable for use in connection with the present invention are red-violet compounds that accumulate naturally in flowers, fruits and some vegetables, most notably cabbage. Useful compounds include anthocyanin, anthocyanidin and their derivatives. These have the chemical formula: 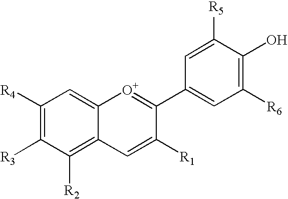
where R₁ is H, O-Sugar or OH, R₂ is OH, O-Sugar or OMe, R₃ is H or OH, R₄ is H, O-Sugar, OH or OMe, R₅ is H, OH or OMe, and R₆ is H, O-Sugar, OH, OMe. (By “sugar” is meant a monosaccharide, oligosaccharide or polysaccharide compound, e.g., glucose, sucrose, etc., or a derivative thereof.) The flavonoid compound may be acylated to produce an ester.

The betalain or flavonoid indicator molecule can be deployed in various ways to create a sensing system useful in accordance with the invention. In one embodiment, the indicator is entrained within a hydrophobic, fibrous matrix such as silicone-treated filter paper, which may safely be brought into contact with food. It is found that even water-soluble betalains and flavonoids are not washed out of the matrix despite exposure to polar compounds; indeed, the treated paper shows indicator activity even following an aqueous wash. Entrainment may be accomplished, for example, by soaking the matrix in a solution of the indicator followed by drying. Other embodiments utilize a fibrous hydrophilic matrix, or a hydrophilic matrix having a hydrophobic coating.

In another approach, the indicator molecule is incorporated within a polymer matrix. This may be achieved quite simply by mixing the indicator with a prepolymer prior to reaction; polymerization entrains the indicator molecule within the polymer matrix, with sufficient surface exposure and/or polymer permeability to facilitate adequate interaction (leading to a visible color change) with food-generated amines. For example, a betalain or flavonoid indicator may be mixed with polystyrene, polyvinylidene chloride and polyvinyl chloride. The polymer may be incorporated within packaging (e.g., as a ribbon wrapped around meat and visible through transparent wrap) or may even define it (e.g., as the wrap itself).

In one experiment, 5 grams of styrene, 0.2 gram of lauryl peroxide and 0.1 gram of beet juice extract were warmed to 85° C. in a water bath and periodically mixed. After several hours the red polymer solidified. Exposure to vapors of amines or ammonia resulted in the characteristic color change for betanin.

Alternatively, the indicator may be covalently bonded to the polymer backbone itself. For example, Reaction 1 may be utilized to bond betanin to a polymer having terminal or distributed hydroxyl functional groups. Similarly, acylation may be employed to bond flavonoids.

The color change exhibited by the indicator can, if desired, be altered for better visibility or for aesthetic or branding (e.g., conformance to a company's trademark color) purposes. This can be accomplished by combining the indicator with a dye that is not adversely affected by pH within the range of interest, or by covering the indicator with a colored film or gelatin. For example, an anthocyanin indicator changes in color from pink to purple with increasing amine concentration. By combining this indicator with a yellow dye (e.g., by simply adding the yellow dye to the anthocyanin mixture prior to entrainment within a fibrous matrix), the visible change will be from orange to green, which may provide better color contrast. So long as the dye is not adversely affected by pH changes within the range of interest—e.g., the dye is largely or substantially pH-insensitive within that range or exhibits a color response at least does not negate the ultimate desired effect of, for example, color contrast—it will be suitable for use in accordance herewith. Alternatively, covering the impregnated fibrous matrix with a yellow film will produce a similar effect.

It will therefore be seen that the foregoing represents a conveniently practiced and versatile approach to sensing food spoilage. The terms and expressions employed herein are used as terms of description and not of limitation, and there is no intention, in the use of such terms and expressions, of excluding any equivalents of the features shown and described or portions thereof, but it is recognized that various modifications are possible within the scope of the invention claimed.

1. An indicator for detecting food spoilage, the indicator comprising a matrix having at least one surface for establishing fluid communication with a food to be monitored, and, immobilized within the matrix, an amine-responsive compound comprising a betalain or derivative thereof or a flavonoid or derivative thereof.

2. The indicator of claim 1 wherein the compound comprises a betalain or derivative thereof.

3. The indicator of claim 1 wherein the betalain comprises an ester of betanin.

4. The indicator of claim 3 wherein the compound has the chemical formula: 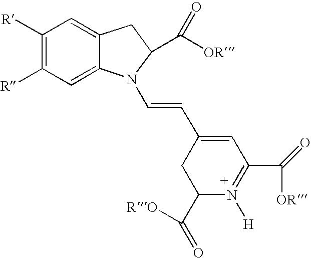
wherein R′ and R″ are hydrogen, hydroxyl, ester, alkyl, aryl, mixed alkyl-aryl groups or GlcO, and R′″ is an alkyl, aryl, or mixed alkyl-aryl group.

5. The indicator of claim 4 wherein R′″ is an alkyl group having up to 20 carbon atoms.

6. The indicator of claim 5 wherein the alkyl group is linear, branched, cyclic or a combination.

7. The indicator of claim 1 wherein the compound comprises a flavonoid or derivative thereof.

8. The indicator of claim 7 wherein the flavonoid comprises an anthocyanin or derivative thereof.

9. The indicator of claim 7 wherein the compound comprises an anthocyanidin or derivative thereof.

10. The indicator of claim 7 wherein the compound has the chemical formula: 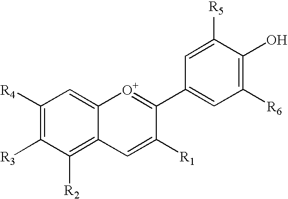
wherein R₁ is H, O-Sugar or OH, R₂ is OH, O-Sugar or OMe, R₃ is H or OH, R₄ is H, O-Sugar, OH or OMe, R₅ is H, OH or OMe, and R₆ is H, O-Sugar, OH, OMe.

11. The indicator of claim 1 wherein the matrix is a hydrophobic or hydrophilic paper or hydrophilic paper with a hydrophobic coating.

12. The indicator of claim 11 wherein the hydrophobic paper is silicone-treated filter paper.

13. The indicator of claim 1 wherein the matrix is a polymer matrix.

14. The indicator of claim 13 wherein the compound is entrained within the polymer matrix.

15. The indicator of claim 13 wherein the compound is bonded to a backbone of the polymer matrix.

16. The indicator of claim 1 further comprising a clear gelatin associated with the matrix.

17. The indicator of claim 1 further comprising a colored gelatin associated with the matrix to improve visibility of a color change exhibited by the indicator in response to amine concentration.

18. The indicator of claim 1 further comprising a secondary dye to improve visibility of a color change exhibited by the indicator in response to amine concentration.

19. A method of making an indicator for detecting food spoilage, the method comprising the steps of:
a. providing an indicator compound comprising at least one of (i) a betalain or derivative thereof and (ii) a flavonoid or derivative thereof; and
b. associating the compound with a matrix having at least one surface for establishing fluid communication with a food to be monitored.

20. The method of claim 19 wherein the compound comprises a betalain or derivative thereof.

21. The method of claim 20 wherein the betalain comprises an ester of betanin.

22. The method of claim 19 wherein the compound comprises an anthocyanin or a derivative thereof.

23. The method of claim 19 wherein the compound comprises an anthocyanidin or derivative thereof.

24. The method of claim 19 wherein the matrix is a hydrophobic or hydrophilic paper or hydrophilic paper with a hydrophobic coating.

25. The method of claim 19 wherein the matrix is a polymer matrix.

26. The method of claim 19 wherein the associating step comprises entraining the compound within the matrix.

27. The method of claim 25 wherein the associating step comprises bonding the compound to a backbone of the polymer matrix.

28. A method of detecting food spoilage using a matrix having, associated therewith, an amine-responsive compound comprising a betalain or derivative thereof or a flavonoid or derivative thereof, the method comprising the steps of:
a. establishing fluid communication between the matrix and a food to be monitored, the amine-responsive compound changing color in response to amines or acids present in or generated by the food; and
b. observing the color change to detect food spoilage.

29. The method of claim 28 wherein the matrix further comprises a colored gelatin associated therewith to improve visibility of a color change exhibited by the indicator in response to amine concentration.

30. The method of claim 28 wherein the matrix further comprises a secondary dye to improve visibility of a color change exhibited by the amine-responsive compound in response to amine concentration.
